(19) United States
(12) Patent Application Publication
(10) Pub. No.: US 2026/0249037 A1
(43) Pub. Date: Aug. 27, 2026

(54) METHOD AND APPARATUS FOR BURST DISPERSION OF MULTIPLE DISTINCT ODORS

(71) Applicant: Young A. Tang, Downey, CA (US)

(72) Inventor: Young A. Tang, Downey, CA (US)

(21) Appl. No.: 19/459,879

(22) Filed: Jan. 26, 2026

Related U.S. Application Data
(63) Continuation-in-part of application No. 19/053,062, filed on Feb. 13, 2025.
(60) Provisional application No. 63/553,000, filed on Feb. 13, 2024.
(60) Provisional application No. 63/749,678, filed on Jan. 26, 2025.

(51) Int. Cl. A61M 21/00; A61L 9/03

(52) U.S. Cl. CPC A61M 21/00; A61L 9/032; A61L 9/035; A61L 2209/111; A61L 2209/131; A61L 2209/133; A61L 2209/134; A61M 2021/0016; A61M 2205/50

(57) ABSTRACT

Disclosed are example embodiments of scent presentation systems and methods. An example system includes multiple scent reservoirs each associated with a distinct odor, an isolation chamber configured to receive vapor from a selected scent reservoir while isolating remaining scent reservoirs, and a positioning mechanism configured to selectively place individual scent reservoirs in fluid communication with the isolation chamber. A scent release mechanism is configured to cause release of vapor from the selected scent reservoir into the isolation chamber, and an airflow mechanism is configured to expel vapor from the isolation chamber toward a user. One or more movable doors are associated with the isolation chamber to permit accumulation of vapor when closed and burst-based expulsion of accumulated vapor when opened. A control module controls selection of scent reservoirs, operation of the scent release mechanism, and coordinated operation of the doors and airflow mechanism according to a programmed scent presentation schedule.

### CROSS-REFERENCE TO RELATED APPLICATIONS

This Patent Application claims priority to U.S. Provisional Patent Application No. 63/749,678, filed on Jan. 26, 2025 and is a continuation in part of U.S. Utility patent application Ser. No. 19/053,062, filed Feb. 13, 2025, which claims priority to U.S. Provisional Patent Application No. 63/553,000, filed on Feb. 13, 2024. The disclosures of the prior Applications are considered part of and is incorporated by reference into this Patent Application.

### TECHNICAL FIELD

The present disclosure relates generally to olfactory stimulation systems and/or scent delivery technologies and, more specifically, to methods and apparatus for controlled, sequential, and burst-based dispersion of multiple distinct odors for therapeutic, cognitive, and sensory applications.

### BACKGROUND OF THE INVENTION

Environmental enrichment has been shown to positively influence cognitive function in a variety of experimental settings, including animal models of cognitive decline. Enrichment protocols may involve, for example and without limitation, introducing novel objects, sounds, colors, smells, social interaction, and/or other sensory stimuli into an environment. Reported benefits of environmental enrichment have included improvements in behavioral and functional outcomes across models associated with neurological and neuropsychiatric conditions, and certain studies have reported measurable behavioral changes following sustained enrichment activities.

Among enrichment modalities, olfactory stimulation has been reported to be associated with cognitive and emotional processing. Unlike many other sensory modalities, olfactory pathways can provide relatively direct neural connections to brain regions implicated in memory and affect. In aging populations, diminished olfactory function has been correlated with cognitive decline and has been reported to precede or accompany memory impairment and certain neurodegenerative conditions. Reduced olfactory performance in older adults has also been associated in the literature with increased risk of adverse health outcomes, including elevated all-cause mortality over subsequent years.

Olfactory stimulation is understood to engage the entorhinal cortex, which is implicated in memory-related processing and may diminish with age and in association with neurodegenerative disease. As entorhinal function diminishes, changes in downstream hippocampal circuitry (including the dentate gyrus and CA3 subfields) may occur, which can interfere with memory formation and retrieval. Accordingly, in some therapeutic contexts, restoring or enhancing olfactory input may promote neural plasticity (e.g., neurogenesis and/or increased neural complexity) within entorhinal-hippocampal pathways, thereby supporting normalization of dentate/CA3 activity and supporting memory-related function.

Despite these observations, environmental enrichment interventions can be expensive, time-consuming, or difficult to maintain over time, particularly where the intervention relies on sustained exercise or other continuous activities. As a result, there remains a need for low-cost, practical, and user-friendly methods and apparatus that can deliver environmental enrichment in a repeatable manner and that may improve or support cognitive function and memory. More specifically, there remains a need for environmental enrichment equipment that leverages olfactory stimulation and that can present multiple distinct odors in a controlled manner suitable for routine use.

### SUMMARY

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

Disclosed are example embodiments of a scent presentation system. The system includes multiple scent reservoirs each associated with a distinct odor, an isolation chamber configured to receive vapor from a selected scent reservoir while isolating remaining scent reservoirs, and a positioning mechanism configured to selectively place individual scent reservoirs in fluid communication with the isolation chamber. A scent release mechanism is configured to cause release of vapor from the selected scent reservoir into the isolation chamber, and an airflow mechanism is configured to expel vapor from the isolation chamber toward a user. One or more movable doors are associated with the isolation chamber to permit accumulation of vapor when closed and burst-based expulsion of accumulated vapor when opened. A control module controls selection of scent reservoirs, operation of the scent release mechanism, and coordinated operation of the doors and airflow mechanism according to a programmed scent presentation schedule.

Disclosed are example embodiments of a method for presenting a scent to a user. The method includes determining, using a control module, a scent to be presented according to a programmed schedule, positioning a scent reservoir associated with the determined scent in fluid communication with an isolation chamber, and heating the scent reservoir to release the scent into the isolation chamber while operating an airflow mechanism to expel the released scent toward the user.

Disclosed are example embodiments of a scent presentation system. The system includes a belt carrying a plurality of scent reservoirs, each associated with a respective odor, and an isolation chamber that is selectively alignable with an individual scent reservoir while remaining reservoirs are isolated. A positioning mechanism indexes the belt to place a selected scent reservoir in fluid communication with the isolation chamber, and a scent release mechanism causes release of scent from the selected reservoir into the isolation chamber. An airflow mechanism directs the released scent toward a user, and one or more doors associated with the isolation chamber permit scent to accumulate when closed and to be expelled as a burst when opened. A control module coordinates belt positioning, scent release, door movement, and airflow according to a programmed scent presentation schedule.

### BRIEF DESCRIPTION OF THE DRAWINGS/FIGURES

FIGS. 1A and 1B illustrate an embodiment as viewed from the front.

FIGS. 2A and 2B illustrate an embodiment as viewed from the rear.

FIG. 3A illustrates a top view of a scent-carrying belt in accordance with some embodiments.

FIG. 3B illustrates a side view of the scent-carrying belt of FIG. 3A.

FIG. 3C illustrates a top view of a scent-carrying belt in accordance with some embodiments.

FIG. 3D illustrates a side view of the scent-carrying belt of FIG. 3C.

FIG. 4 is an exploded view of an embodiment.

FIG. 5 illustrates a process of presenting scents to a user, according to an embodiment.

FIG. 6 illustrates an ultrasonic diffuser according to an embodiment.

FIG. 7 illustrates a nebulizing diffuser according to an embodiment.

FIG. 8 illustrates an evaporative diffuser according to an embodiment.

FIG. 9 illustrates a heat diffuser according to an embodiment.

FIG. 10 illustrates an ultrasonic nebulizer according to an embodiment.

FIG. 11 illustrates a mesh nebulizer according to an embodiment.

FIG. 12 illustrates a jet nebulizer according to an embodiment.

FIG. 13 illustrates a system for scent dispersion, according to an embodiment.

FIG. 14 illustrates a system for scent dispersion, according to an alternative embodiment.

Further embodiments, features, and advantages of the present invention, as well as the operation of the various embodiments of the present invention, are described below with reference to the accompanying drawings.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION OF THE INVENTION

Example embodiments are now described with reference to the figures, where like reference numbers indicate identical or functionally similar elements. While particular configurations and arrangements are described for purposes of illustration—including embodiments that use a belt carrying multiple scent reservoirs to sequentially position different scent source materials adjacent an isolation chamber for burst-based release—other configurations and arrangements may be employed without departing from the spirit and scope of the disclosure. For example, in addition to odor-infused wax embodiments, the scent source material may comprise one or more essential oils (including pure oils or oil-containing mixtures) and may be released using any suitable scent release mechanism, such as controlled heating, evaporative release, diffusion, ultrasonic atomization, nebulization, or combinations thereof, optionally in coordination with one or more doors that enable accumulation of scent within a chamber and subsequent burst expulsion toward a user. Accordingly, the disclosed concepts may be implemented in a variety of other systems and applications consistent with the teachings herein.

To address this challenge, the embodiments described herein provide a scent presentation device configured to selectively release distinct odors in a controlled, sequential manner while limiting accumulation of residual scent in the surrounding environment. The device may isolate a selected scent source material, cause release of the scent into an isolation chamber, and direct the released scent toward a user according to a programmed schedule before transitioning to a next scent. By enabling precise control over scent release, isolation, and clearing between successive odors, the disclosed embodiments facilitate reliable delivery of multiple distinct scents for therapeutic, cognitive, and sensory applications.

The systems and apparatuses described herein may be used for multiple purposes, including the therapeutic purposes noted above. The scent presentation device/system (or dispersion device/system) described herein can use various types of mechanisms such as a diffusion mechanism, scent release mechanism, nebulization mechanism, evaporative mechanism, or a combination thereof, for example and without limitation. Hereinafter, the term “scent release mechanism” can include one or more of the above mechanisms.

The process of olfactory therapy can require sequential detection by a patient of a number of novel and distinct odors. This has historically been either a manual process (e.g. sniffing from sample vials) or a somewhat invasive process (e.g., using masks or nasal tubes). The challenge lies in delivering distinct odors to a patient from a distance, without oversaturating the environment with odorant. Oversaturation would make it difficult to clear one distinct odor before starting the next.

In some embodiments, the description herein discloses embodiments of a device that selectively releases distinct odors in a controlled, sequential manner while minimizing environmental oversaturation. In some embodiments, scent source materials are embodied as odor-infused wax, which exhibits a substantial differential in odor transmission rate between solidified and heated states. Multiple scent reservoirs, each associated with a different scent source material and resulting in a respective odor, may be positioned within the device and individually selected for activation. For a selected reservoir, the scent source material may be heated or otherwise energized to release scent into an isolation chamber, where the released scent may be exposed to a controlled air current and transmitted to a user or patient at a distance. After a predetermined interval, release of the scent may be reduced or terminated—such as by cooling, de-energizing, or isolating the scent source material—before transitioning to a next reservoir and repeating the process. The disclosed embodiments may be used to provide olfactory therapy and related sensory stimulation that may be useful for treating memory degradation and other neurological conditions. As used herein, the terms “vapor” and “vaporization” are employed broadly to refer to any facilitated release of scent-bearing molecules, whether or not such release involves a discrete phase change to a gaseous state.

Embodiments of the apparatus and processes include a number of features, listed here and detailed below, but are not limited to these features:

Selective exposure, to a current of air, of a single scent among a multitude of scents; and repeating for each of a sequence of scents.

Selective exposure to the environment of a single scent among a multitude of scents; and repeating for each of a sequence of scents.

Selective heating and melting of a wax piece, with its incorporated scent source material, from among a multitude of wax pieces; and repeating for each of a sequence of wax pieces.

A series of scent reservoirs containing odor-infused wax pieces, arranged on a closed-loop belt with the opening of each reservoir facing towards the center of the loop.

A membrane to seal off the opening of each scent reservoir, which allows odor to pass through but prevents wax from passing through.

A pair of drums, around which the belt of scent reservoirs is wrapped. An upper drum engages with features on the belt and rotates to function as a drive wheel to move the belt around the two drums. A lower drum is hollow, does not rotate, and has an opening on its bottom face to expose the opening of a single reservoir to the center of the lower drum.

A fan, positioned at the rear of the lower drum, which blows air through the lower drum, over the opening of a single scent reservoir, and out the front of the device.

A heating element, which raises and lowers to surround the single scent reservoir that is exposed to the center of the lower drum, in order to heat the contents of the scent reservoir.

An enclosure, which isolates all the scent reservoirs from the user environment except for the reservoir that is presently aligned with the opening on the bottom of the lower drum.

In some embodiments, each scent reservoir on the belt includes an outlet region configured to form a temporary seal with a corresponding inlet region of the lower drum and/or isolation chamber when the reservoir is aligned with the opening. The seal may be formed using one or more of an elastomeric gasket, a compressible lip, a tapered interference fit, a magnetic coupling, or a clamped interface, thereby limiting lateral leakage of vapor and reducing unintended mixing with adjacent reservoirs. In such embodiments, the isolation chamber may receive vapor substantially only from the selected reservoir while the remaining reservoirs remain physically and/or pneumatically isolated from the airflow chamber.

In some embodiments, the belt is indexed to discrete “presentation positions” such that each reservoir is aligned with the opening within a tolerance range determined by the control module. The indexing may be achieved using detents, belt teeth, sprockets, stepper motor positioning, and/or closed-loop feedback from the belt position sensor. The control module may confirm alignment before enabling heating, diffusion, nebulization, and/or door opening, thereby improving repeatability of burst magnitude and reducing variability between sessions.

In some embodiments, the belt includes intermediate “buffer zones” between reservoirs that are configured to reduce carryover. For example, a buffer zone may include an absorbent pad, porous polymer, or sacrificial neutralizing medium that captures or neutralizes residual vapor near the belt surface during belt movement. Additionally or alternatively, the control module may command an active purge cycle (e.g., fan on with doors open and heating off) after belt movement and before the next reservoir is activated.

A door, which opens the enclosure to allow for installation and replacement of the belt and for maintenance of other internal components.

One or more control modules, programmable to create specific schedules and sequences of scent delivery.

The scent presentation apparatus described herein allows for a multi-pronged approach to scent delivery and scent isolation, enabling environmental isolation, airflow isolation, and thermal isolation. A device could selectively expose one scent at a time to the user environment using one or more of or a combination of an isolation chamber, directed air stream, and directed heat source. By combining scent isolation, environmental isolation, airflow isolation, and thermal isolation, the apparatus described here provides the necessary combination of fast detection, fast clearing, clear differentiation, and minimal accumulation of scents.

In an embodiment, the airflow chamber may include doors that may be opened or closed in a manner controlled by the control module(s), such that the chamber is closed during some or all of the initial scent release process. In this manner, a scent is accumulated and concentrated in the closed chamber. After a predetermined time, the chamber may be opened and the fan or other airflow mechanism is activated to push the concentrated scent out of the device.

Additional detail is now provided with respect to the figures presented herewith.

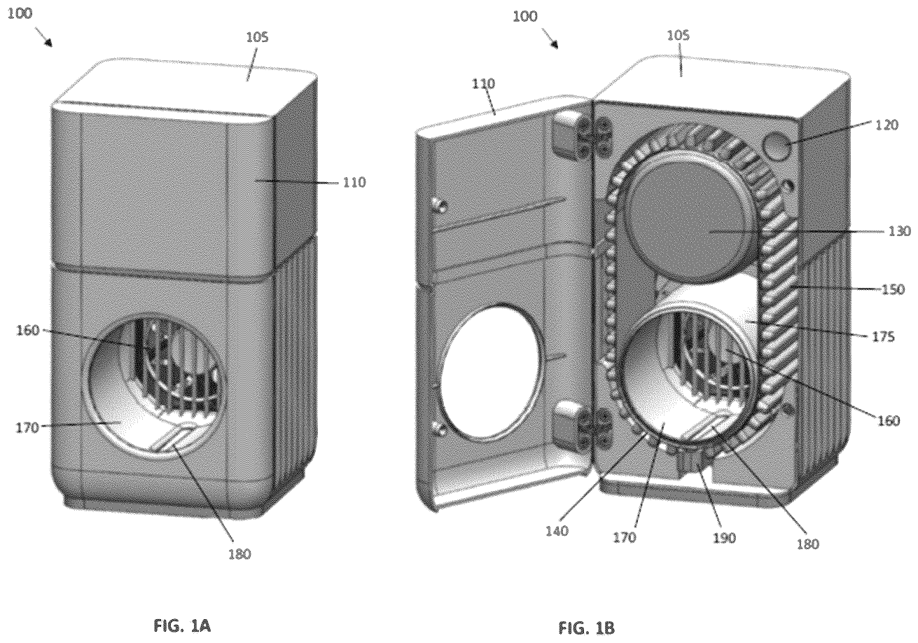

FIG. 1A shows a scent presentation device 100 for providing a series of scents, according to an embodiment. The device may include a rear enclosure 105 and an access door 110 that may be opened as necessary for maintenance purposes. The device 100 may include an airflow chamber 170 which is a volume of space formed by the interior of a lower drum, as will be described in greater detail below. At the bottom of airflow chamber 170 is an opening 180 that exposes the contents of a scent reservoir. The device 100 may include a series of scent reservoirs, at least some of which may provide a respective distinct scent. For a scent reservoir positioned beneath the opening 180, vapor of the scent may be released through the opening 180 and into the airflow chamber 170. A fan 160 at the rear of device 100 may then blow the vapor out of the airflow chamber 170. As used, the device 100 may be used to direct the vapor to a user. The airflow chamber 170 also serves as an isolation chamber, in that a single scent is introduced into the chamber at any given time. The scent is cleared from the chamber by operation of the fan 160 before any further scent is introduced.

Note that in some applications, the user may be a patient for which the series of scents may have therapeutic value.

Some of the internal components of device 100 are shown in FIG. 1B according to an embodiment. These components may include a start button 120. These components may further comprise a closed-loop belt 140. The belt 140 may be a band to which the series of scent reservoirs 150 is attached. Each reservoir may be spaced apart from each other on belt 140, and each reservoir may contain a different scent. In some embodiments, no two adjacent reservoirs contain the same or similar scent. The band may be made of a durable yet flexible material, discussed in greater detail below. In the illustrated embodiment, the belt 140 may be moved by a rotatable upper drum that effectively serves as a drive wheel 130. The belt 140 may therefore be moveable in a looping path around a portion of lower drum 175. The lower drum 175 has an opening 180 at the bottom in the illustrated embodiment. Belt 140 may also be moved by lower drum 175. Although not shown, in one embodiment, device 100 may comprise a single rotatable drum such as drive wheel 130. In this embodiment, belt 140 is configured to partially or fully wrap around the single rotatable drum. Other than the two-drum aspect of device 100, all other features and functions as described herein can be implemented in the single drum device.

In operation, drive wheel 130 is configured to move the belt 140 in a manner that positions one of the scent reservoirs 150 near the opening 180 and in proximity to a heater block 190. In some embodiments, drive wheel 130 is configured to rotate the belt 140 such that one of the scent reservoirs 150 is located below the opening 180. Once one of the scent reservoirs 150 is positioned below the opening 180, the motion of belt 140 may be paused for a desired duration. In this way, heater block 190 heats the scent reservoir 150, thereby causing a scent embedded in the reservoir 150 to be released for a determined amount of time. In some embodiments, the heater block 190 may be composed of metal, metal composite, ceramics, and/or other materials in combination. In an embodiment, the heater block 190 may be raised or moved into a position closer to the now stationary scent reservoir 150, and heat may be applied by the heater block 190. In an embodiment, the heater block 190 may be recessed so that it may surround the scent reservoir 150 to provide efficient heat transfer.

In some embodiments, a heating rod (not shown) can be implemented in place of heater block 190. The heating rod can be positioned near or just above the opening 180. Similar to heater block 190, heating rod may be composed of metal, composite, ceramics, or a combination thereof.

In an embodiment, the scent reservoir 150 holds a piece of wax that incorporates a scent source material, such as a scented oil or other scent-bearing material. Heating the wax allows the scent within the scent-bearing material to be released. In some embodiments, the scent-bearing material may also be heated when the wax is heated. When heated, the scent-bearing material may undergo a heat-induced change in one or more of its material characteristics, thereby releasing the scent. In some embodiments, the scent-bearing material is the wax itself. When heated, the wax can partially or fully melt and cause the scent to be released. Scent or vapor from the wax and/or the scent-bearing material then enters the interior of the lower drum 175 when heat is applied for a predetermined heating interval. The interior of the lower drum 175 is shown in FIGS. 1A and 1B as airflow chamber 170. The fan 160 may be activated to push the vapor through the airflow chamber 170 and out of the device 100. The device 100 may be positioned such that the vapor is directed towards a user.

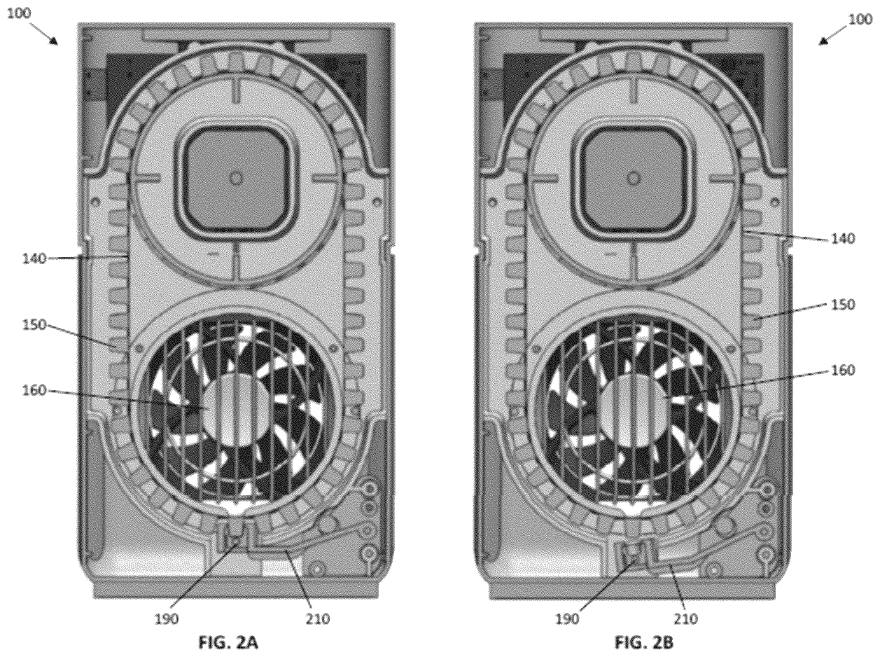

FIGS. 2A and 2B show the rear of device 100, according to an embodiment. In both figures, belt 140 is shown having a series of scent reservoirs 150 attached thereto. In an alternative embodiment, the scent reservoirs may be integrated or embedded into the belt. The fan 160 is shown in a position to draw air in from the back of device 100. FIG. 2A shows a heater block 190 positioned to heat one of the scent reservoirs 150. In some embodiments, the heater block 190 can be positioned such that it encompasses a portion of reservoir 150. The heater block 190 can be positioned by a connected lever arm 210. Alternatively, the heater block 190 can be positioned by other mechanical means such as an axial actuator (not shown) positioned below reservoir 150.

After a heating interval has been completed, the lever arm 210 and heater block 190 may move away from the scent reservoir as shown in FIG. 2B. In this way the wax piece in the scent reservoir may be allowed to cool. In an embodiment, as the wax piece cools, the belt 140 may rotate so that the cooling scent reservoir moves away and a different scent reservoir may be put into place for heating.

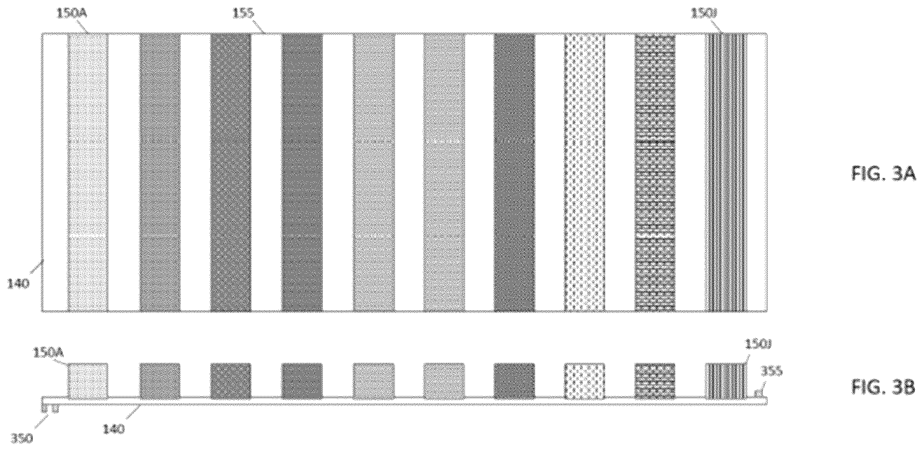

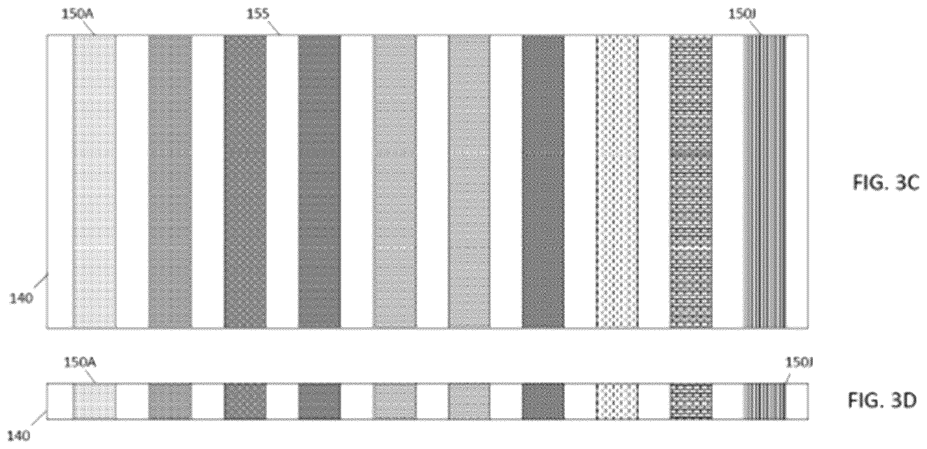

FIG. 3A illustrates a section of an embodiment of belt 140 having a plurality of scent reservoirs 150A through 150J. While this embodiment shows ten scent reservoirs on the belt, other embodiments may have more or fewer. Each reservoir can contain a different scent. Belt 140 may alternatively contain one or more repeating scents. In some embodiments, no two adjacent reservoirs contain the same or similar scent. Scent reservoir 150 may be disposed on a surface of belt 140 as illustrated in FIG. 3B. Additionally, belt 140 includes scent free zones 155 disposed between adjacent scent reservoirs 150. In some embodiments, scent free zones 155 can contain or be infused with scent neutralizers. Each scent free zone 155 can contain different scent neutralizers configured to neutralize a specific scent. For example, one of the free zones 155 can have neutralizer specifically formulated to neutralize a base scent of a fragrance pyramid. Another free zone 155 can have a different neutralizer specifically formulated to neutralize a top scent. Scent neutralization is discussed further below.

Belt 140 may be made of two or more belt sections that each include attachment features 350 and 355 at each end of the belt section. In this way belt sections can be fastened to each other to form a continuous band. Attachment features 350 and 355 may be disposed on one of the surfaces of each belt section. Alternatively, attachment features 350 and 355 may be disposed at the side edge at the very end of each belt section. Attachment features 350 and 355 may be a male-female fastening feature, a hook and loop fastening feature, or other fastening means such as magnets and adhesives.

In an embodiment, the belt 140 may be made of four belt sections connected end-to-end using the attachment features, forming a closed loop. The belt sections may be made of a plastic or polymeric material, such as polypropylene, high-density polyethylene, or high impact polystyrene, as examples without limitation.

In another embodiment, the belt may not be a loop but may instead be linear. In this case, an alternative mechanism (not shown) may be used to move the belt back and forth as necessary to position its scent reservoirs in proximity to a heating element.

FIGS. 3C and 3D illustrate an embodiment of belt 140 where the plurality of scent reservoirs are embedded within belt 140. Rather than being raised above the surface of belt 140, scent reservoirs 150 of this embodiment are integrated within belt 140.

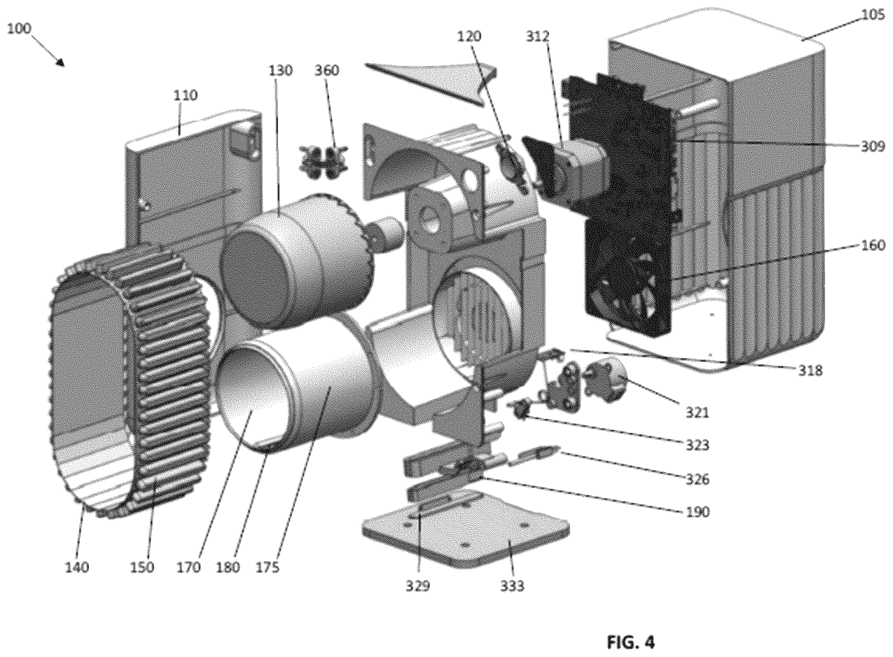

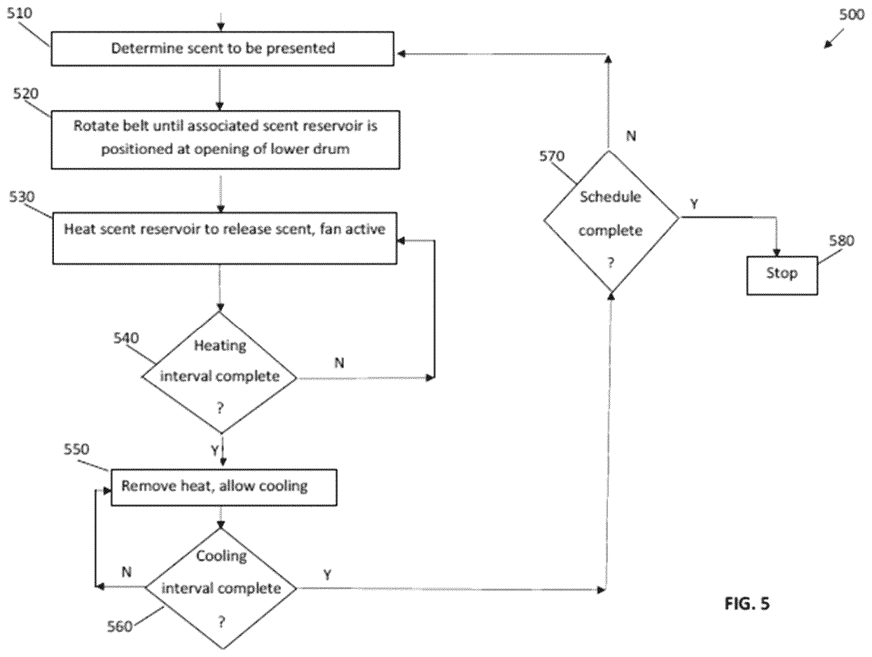

FIG. 4 shows device 100 in an exploded view. Belt 140, with its scent reservoirs 150, are mounted on a motorized drive wheel 130 and on the stationary lower drum 175. The motion of the drive wheel 130 and the belt 140 may be determined by a drive wheel motor 312. The drive wheel motor 312 may be controlled by a control module. The control module may be implemented as logic embodied on a main printed circuit board (PCB) 309. In various embodiments, this logic may be implemented in hardware, firmware, software, or a combination thereof. This logic may control when the drive wheel turns, how far it turns, when it pauses, and the length of the pause. This logic may also control the duration of each heating interval and/or the intensity (i.e., temperature) of the heating process. In this way, the logic of PCB 309 may specify the particular scent reservoir 150 that is heated, the duration and/or temperature of the heating, the duration of the cooling, and the sequence of scent reservoirs 150 (and the corresponding sequence of scents) that are presented to a user. The logic may also specify the higher-level scheduling of operation of device 100, i.e., the timing and duration of a sequence of scent presentations. For example, the logic may specify that scent presentations take place every night during hours when the user is normally in bed. In another embodiment, the logic may be responsive to biometric sensors that measure physiological parameters of the user, so that scent presentation only takes place when these parameters indicate one or more particular stages of sleep.

PCB 309 can also include logic to control the position of the heater block arm 210 (cam) or the axial actuator (not shown) based on a scent presentation schedule, heating interval, and/or intensity. For example, during a scent presentation mode, PCB 309 may include logic to move the heater block 190 into place and turn on heater block 190. During a break period between scent presentations, PCB 309 may include logic to move the heater block 190 away from scent reservoir 150 and/or turn off the heater block 190.

To facilitate control of the belt 140, a belt position sensor 318 may be used to detect the position of the belt 140 and to provide this information to the logic of PCB 309. The position of belt 140 may be sensed by an optical and/or mechanical process. For example, the belt position sensor 318 may optically detect physical or identifying markers on the belt 140 and/or on the scent reservoirs 150 to determine the position of the belt. Such identifying markers can be a bar code, QR code, NFC (near field communication) chip, NFC tag, RFID (radio frequency identifier) tag, or low energy Bluetooth beacon associated one or more scent reservoirs 150 or each belt 140, for example and without limitation. The identifying marker can identify one or more of: the position of a scent reservoir with respect to the isolation chamber 170; the scent type (e.g., peach, vanilla, cinnamon, mint) of each scent reservoir on belt 140; the scent identifying number, which can also provide the scent type and other characteristics (e.g., recommended neutralizer); and the order of the scent as positioned on belt 140. Alternatively, the belt position may be determined by tracking the degree and direction in which the belt has moved with respect to a known reference point. In an embodiment, the logic of PCB 309 may be programmable by a manufacturer, a medical care provider, and/or a user. The interface for such a programming process may be wired or wireless and use any communications protocol known to persons of ordinary skill in the art. In an embodiment, the programming may be performed through an application running on a user's computing device, such as a personal computer, laptop, tablet, smartphone, or other user device.

As discussed above, a wax piece may be heated by a heater block 190. The heater block 190 may be covered on its underside by an insulated heater block cover 329. Further insulation may be provided by heater block arm 210. The heater block 190 may be heated by a heating element, shown here with a thermistor and thermostat. These three components are shown collectively as assembly 326. The heater block 190 may be moved into and out of position by a heater block motor 321. Positioning of the heater block 321 may be further controlled by a limit switch 323.

Operation of device 100 includes the following actions, according to an embodiment. The logic of PCB 309 may first be programmed by a user, caregiver, retailer, or manufacturer to specify a sequence or rotation of particular scents that device 100 is to present. The programming may also specify the duration of presentation of each scent and/or a time interval between scents.

Different scents in the programmed sequence correspond to respective scent reservoirs 150 on belt 140. The sequence of scents therefore corresponds to a series of scent reservoirs 150 that are to be positioned below opening 180. This in turn corresponds to a series of positions of belt 140, where each position corresponds to the appropriate scent reservoir 150 positioned below the opening 180. A particular belt position may therefore be identified by the logic running on PCB 309. Under control of this logic, drive wheel motor 312 may rotate the drive wheel 130 to position belt 140 so that the desired scent reservoir 150 is beneath the opening 180 of the lower drum 175. Drive wheel 130 and belt 140 may then halt. The required positioning of the belt 140 may be facilitated by the belt position sensor 318.

Once stationary, the scent reservoir 150 is in proximity to the heater block 190, and may then be heated by heater block 190, by raising the lever arm 210 so that the heater block 190 is adjacent or sufficiently close to the scent reservoir 150, and activating heating element assembly 326. This melts, at least partially, the wax piece of scent reservoir 150 and vaporizes some of the scent source material therein, releasing the vapor into airflow chamber 170.

In an embodiment, the scent reservoir 150 may include a membrane or other barrier (not shown) that allows the vapor to pass out of the scent reservoir 150 while maintaining the wax in place. The fan 160 may push air over the opening 180 to move the scent out of device 100 and toward a user.

Once the scent has been delivered for a time interval specified by the programming, the lever arm 210 may lower, moving the heater block 190 away from the scent reservoir 150, which may then be allowed to cool. In an embodiment, the lever arm 210 may be spring loaded to raise the heater block 190, and a rotating cam may be used to lower the lever arm 210, moving it from the scent reservoir 150. A next scent (and associated scent reservoir 150) may be specified by the programming, and the process may be repeated for this latter scent reservoir, and for any further scents and scent reservoirs. The motion of the lever arm 210 and heater block 190 towards and away from the stationary scent reservoir 150 may be actuated by a mechanism connected to heater block motor 321 and comprising one or more cams, as would be understood by a person of ordinary skill in the art.

In the illustrated embodiment, the above components are housed in a two-piece chassis comprising a rear enclosure 105 and an access door 110, which may be connected by one or more hinges 360. The device may also include a base 333. The base 333 may be made of one or more of a heat insulating material, a vibration-absorbing material, and/or a sound deadening material. Such a base 333 would facilitate placement of device 100 in a home or professional setting if desired.

In an embodiment, the apparatus may include doors at the ends of the airflow chamber 170. The doors may be closed during some or all of the heating process of a reservoir. In this manner, the scent emanating from the heated reservoir can be concentrated in the closed chamber. After a predetermined time, the doors may be opened and the concentrated scent may be pushed out of the chamber by the fan, resulting in a burst-like release of the scent. The opening and closing of the doors may be controlled by one or more motors. The door motor(s) may be actuated under the control of the control module. In an embodiment, the length of time during which the chamber is closed may be the same for all reservoirs; in an alternative embodiment, different scents and reservoirs may have different closure intervals. For example, when a reservoir having a milder scent is being heated, the chamber may be closed for a longer duration than for other scents, and when a reservoir having a stronger scent is being heated, the chamber may be closed for a shorter duration. Further, in an embodiment, this process may be repeated two or more times during a single heating process of a reservoir. In such an embodiment, there may be two or more releases, or bursts, of a given scent during a single heating of a reservoir. In an embodiment, after one or more bursts the chamber doors may remain open while heating of the reservoir continues and fan continues to operate.

In some embodiments, the control module is configured to generate one or more burst events for a selected reservoir by controlling a coordinated sequence that includes: (i) closing the doors to allow vapor accumulation, (ii) enabling the scent release mechanism for an accumulation interval, (iii) opening the doors, and (iv) operating the airflow mechanism for a burst expulsion interval. The control module may repeat the sequence two or more times while the selected reservoir remains aligned with the opening, thereby producing multiple bursts of the same odor from a single reservoir activation.

In some embodiments, “burst shaping” is performed by controlling one or more of door opening speed, door open area, fan speed profile, and/or pressure equalization vents, such that a burst may be configured as a short high-intensity pulse, a stepped pulse train, or a tapered release. For example, the control module may command a first fan speed during an initial pulse and a second lower fan speed during a trailing interval to reduce overshoot and improve user comfort. Burst parameters may be set globally or per scent based on scent strength, pyramid note classification, user preference, room size, humidity, or other environmental parameters.

Note that the above embodiment includes a belt mechanism for manipulating scent reservoirs and an evaporative process for releasing scents from the reservoirs. Other embodiments may have alternative mechanisms for initial scent release from a scent source.

For example, other embodiments can use various types of mechanisms such as a diffusion mechanism, scent release mechanism, nebulization mechanism, evaporative mechanism, or a combination thereof, for example and without limitation. Hereinafter, the term “scent release mechanism” can include one or more of the above mechanisms.

In some embodiments, one or more scent reservoirs on the belt contain a liquid scent-bearing material comprising an essential oil, a blend of essential oils, and/or a mixture of essential oil and a carrier (e.g., water, alcohol, glycol, or other diluent). The reservoir may be implemented as a replaceable cartridge having a sealed body and a controlled outlet, such as a valve, septum, pierceable membrane, or capillary restrictor, which inhibits leakage during belt movement and allows dispensing only when the reservoir is positioned for presentation.

In some embodiments, the reservoir includes a wick, porous insert, or absorbent matrix that retains the essential oil while exposing a controlled surface area to airflow and/or to the selected scent release mechanism. For example, an evaporative embodiment may direct airflow across the exposed wick surface, while a heating embodiment may heat the reservoir body and/or wick to increase volatilization. In a nebulizing or ultrasonic embodiment, the reservoir may feed liquid to a dispersion interface (e.g., ultrasonic membrane, jet orifice, mesh, or transducer chamber) via gravity, capillary action, pump, or microfluidic feed channel.

In some embodiments, the control module compensates for different viscosities and volatilities across essential oils by adjusting at least one of: heater temperature, heater duty cycle, accumulation interval, burst interval, fan speed, and/or purge duration. Additionally, or alternatively, a reservoir may include a temperature sensor, liquid level sensor, or conductivity sensor to provide feedback for maintaining consistent output between different oils and blends.

The scent presentation device/system can use ultrasonic diffusers, which employ ultrasonic vibrations to create a fine mist from a mixture of water and essential oils. The mist is then released into the air, adding humidity and releasing the scent.

The scent presentation device/system can use nebulizing diffusers, which work by using pressurized air to atomize pure essential oils, turning them into a fine mist without the use of heat or water.

The scent presentation device/system can use an evaporative diffuser, which uses a fan to evaporate the essential oils from a pad or filter, or simply from oil droplets exposed to the airstream (with no carrier medium). As the oil evaporates, the fan blows the scent into the air. Evaporative diffusion may be suitable for personal use or in volumetrically smaller settings.

The scent presentation device/system can use a heat diffuser to gently heat essential oils, causing them to evaporate and disperse their scent into the air.

The scent presentation device/system can use various types of nebulizers. The scent presentation device/system can use an ultrasonic nebulizer, which uses high-frequency ultrasonic vibrations to generate a mist from a liquid medication. Such a system may comprise a piezoelectric transducer, which converts electrical energy into mechanical vibrations. These vibrations then propagate through the liquid, creating aerosol droplets. The efficiency of ultrasonic nebulizers in creating a fine mist rapidly is notable, although the thermal effect of the process on medication stability warrants consideration for thermolabile substances.

The scent presentation device/system can use a mesh nebulizer, which uses a mesh or membrane with precisely sized openings, through which the liquid (e.g., oil, water-oil mixture) is forced under pressure, creating a fine mist. This category of systems can be subdivided into active and passive systems. Active mesh nebulizers employ a vibrating element to propel the liquid through the mesh, whereas passive systems rely on the user's inhalation to draw the liquid across. The precision engineering of the mesh ensures consistent droplet size, making these devices highly efficient for drug delivery with minimal residue.

The scent presentation device/system can use a jet nebulizer, which operates on the principle of pressurized air flowing through a narrow orifice, converting a liquid medication into aerosol droplets. This process involves a Venturi system, where the high velocity of air creates a negative pressure that draws the liquid medication from the reservoir, atomizing it into a breathable mist.

With any of these alternative mechanisms, the scent may be initially released into a closed airflow chamber where it may be allowed to accumulate. After a predetermined period, the airflow chamber may be opened and the accumulated scent presented in a burst to a user through use of a fan or other airflow mechanism.

A process 500 for presenting scents to a user is illustrated in FIG. 5, according to an embodiment. At 510, a scent is identified for presentation. This scent may be determined by the logic programmed in the control module. At 520, the belt is rotated until the scent reservoir associated with the determined scent is exposed in the isolation chamber. In some embodiments, belt 140 is rotated until the selected scent reservoir is in proximity to the opening of the lower drum 175. At 530, the scent reservoir is heated by the mechanism described above. This releases the determined scent into the lower drum (i.e., the isolation chamber). Activation of the fan pushes the scent out of the apparatus.

In some embodiments, belt 140 is stationary and an isolation chamber (i.e., lower drum 175) can be moved to a desired scent reservoir of belt 140 to selectively isolate the scent reservoir for the scent release/presentation process. This can include moving a heat source (i.e., heater block 190) to the desired scent reservoir of belt 140. Alternatively, the heat source can be stationary and can be coupled to the moveable isolation chamber via a flexible duct.

In some embodiments, the belt carries multiple liquid reservoirs, while a centralized dispersion module includes a dispersion reservoir configured to receive a metered quantity of liquid from a selected belt reservoir. The transfer may be performed using a micro-pump, peristaltic pump, gravity feed, or valve-controlled dosing, after which the dispersion module atomizes or evaporates the transferred liquid using one or more of the aerosolization mechanisms described herein. This architecture can reduce duplication of dispersion hardware while maintaining the ability to select among multiple essential oil reservoirs.

In some embodiments, after completion of a presentation cycle, the centralized dispersion module performs a purge/clean cycle that reduces cross-scent contamination, including one or more of: (i) draining residual liquid, (ii) flushing with a neutral liquid, (iii) running an air-only purge, and/or (iv) dispensing a neutralizer. The purge/clean cycle may be automatically initiated between scents and may be controlled based on scent type, oil class, and/or prior burst intensity.

The release of the scent continues until the heating interval is completed. The length of the heating interval may be determined by the programming of the control module and may be unique to the determined scent or may be the same for all scents. A determination may be made at 540 as to whether the heating interval has been completed. In some embodiments, in making this determination, the control module may monitor an elapsed heating duration using a timer or sensor input and generate a control signal that either maintains operation of the heating element or initiates termination of the heating based on the monitored duration. If not, heating continues at 530.

If the heating interval has concluded, then at 550 the heating ends and the heating element is removed from the scent reservoir. This allows cooling of the scent reservoir. The length of the cooling interval may be determined by the programming of the control module, and may be unique to the determined scent or may be the same for all scents. A determination may be made at 560 as to whether the cooling interval has completed. In some embodiments, in making this determination, the control module may monitor an elapsed cooling duration or a temperature associated with the scent reservoir and generate a control signal that either maintains a cooling state or enables advancement to a subsequent scent presentation. If not, cooling continues at 550.

The cooling interval can be based on one or more of the size of the room in which the device is operating, characteristics of the scent (e.g., base, middle, or top notes as would be mapped on the fragrance pyramid), temperature and/or humidity in the room; and altitude (which may be determined by user's location, using, for example, global positioning coordinates or the user's internet protocol address), the device's fan speed and/or fan capacity (e.g., cubic feet per minute), user's input, and the distance between the device and the user in operation.

When the cooling interval has completed, a determination is made at 570 as to whether the entire schedule of scents has been completed. In some embodiments, evaluating whether the schedule has been completed includes comparing a current scent presentation index or identifier to stored schedule data and generating a control signal that either initiates another scent selection or terminates operation of the device. If not, the process returns to 510, where another scent is determined by the programming for presentation. The process 500 may repeat, so that a sequence of scents is presented to the user. Once this schedule has completed, as determined by the programming at 570, the process may stop at 580.

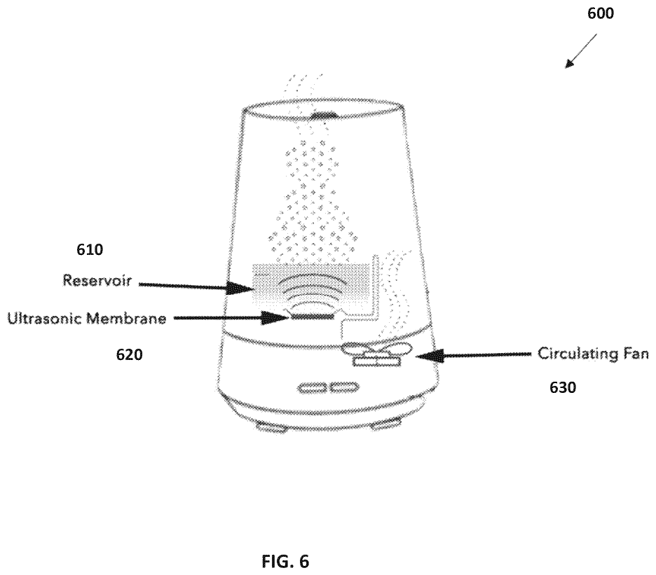

A scent presentation device/system can use an ultrasonic diffuser as a scent release mechanism, employing ultrasonic vibrations to create a fine mist from essential oil(s) or a mixture of water and essential oil(s). The mist may then be released into the air, adding humidity and dispersing the scent. An embodiment of an ultrasonic diffuser is shown in FIG. 6. A scent reservoir 610 may contain a scent bearing liquid, such as a mixture of water and essential oils. An ultrasonic membrane 620 is made to vibrate to create acoustic waves in the liquid, creating a mist that is propelled out of the device by a forced air mechanism such as a fan 630.

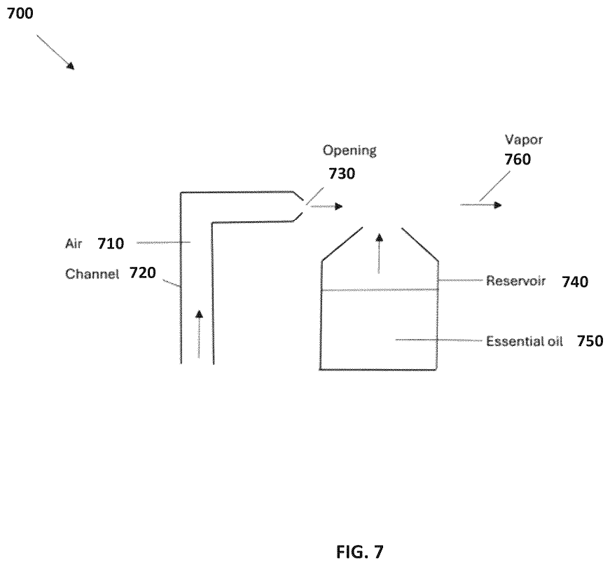

The scent presentation device/system can alternatively use a nebulizing diffuser as a scent release mechanism, which works by using pressurized air to atomize essential oil(s), either pure or in a mixture, turning them into a fine mist without the use of heat or water. An embodiment is shown in FIG. 7. Here air 710 may be propelled through a channel 720 and an opening 730. A reservoir 740 may hold a scent-bearing liquid such as an essential oil or mixture thereof (250). The propelled air results in lower air pressure at the opening of reservoir 740, drawing evaporated oil 750 from the reservoir 740 in the form of vapor 760, which is then dispersed.

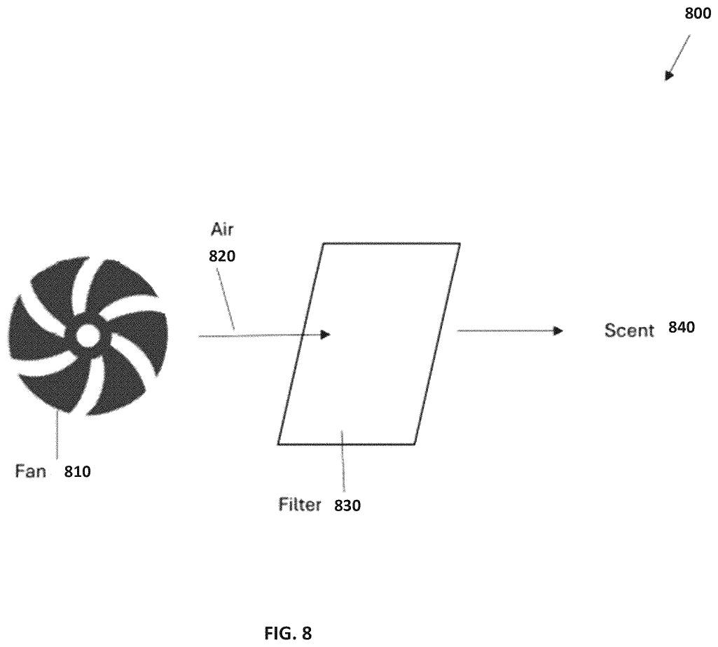

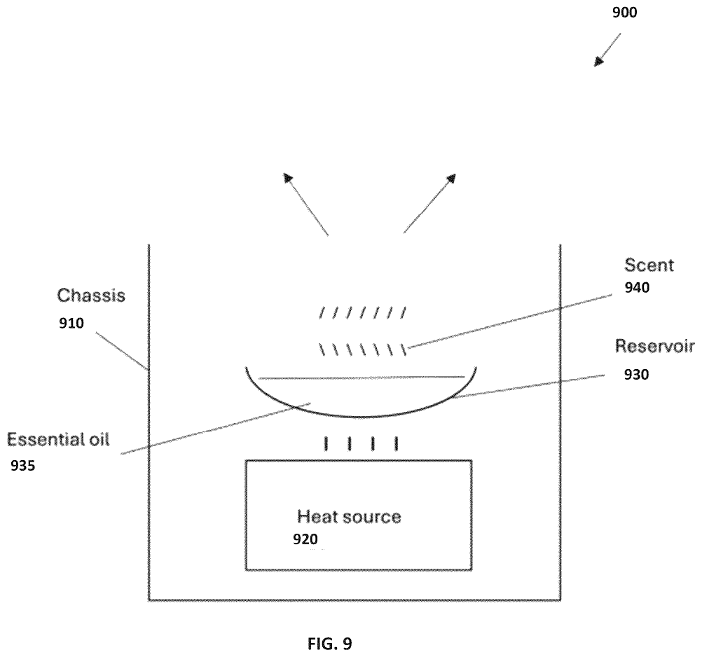

The scent presentation device/system can alternatively use an evaporative diffuser as a scent release mechanism, as illustrated in FIG. 8 according to an embodiment. This embodiment may use a fan 810 to move air over a pad or through a filter 830 where the pad or filter holds one or more essential oils or a mixture thereof for example. This may evaporate the essential oils from the pad or filter. Alternatively, the air stream may evaporate oil droplets exposed to the airstream (with no carrier medium). In any case, as the oil evaporates, the fan 810 may blow the scent into the air. Evaporative diffusion may be suitable for personal use or in volumetrically smaller settings, for example.

The scent presentation device/system can use a heat diffuser as the scent release mechanism, to heat essential oil(s), causing them to evaporate and disperse their scent into the air as shown in the embodiment of FIG. 9. A heat source 920 is shown in a chassis 910, and heats a reservoir 930 containing a scent bearing liquid such as an essential oil or mixture thereof (435). The resulting vapor with scent 940 is thereby released.

The scent presentation device/system can use various types of nebulizers as scent release mechanisms.

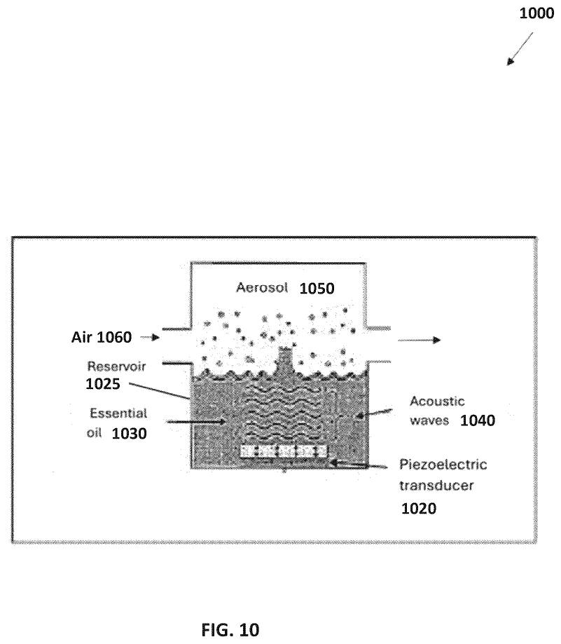

The scent presentation device/system can use an ultrasonic nebulizer as a scent release mechanism as shown in FIG. 10 according to an embodiment. Here, a piezoelectric transducer 1020 may create high-frequency ultrasonic vibrations in an essential oil or a mixture thereof (530) in a reservoir 1025. Resulting acoustic waves 1040 generate a mist or aerosol droplets 1050 from the liquid 1030. The aerosol 1050 may then be pushed out of the mechanism by an air current 1060. The efficiency of ultrasonic nebulizers in creating a fine mist rapidly is notable, although the thermal effect of the process on medication stability warrants consideration for thermolabile substances.

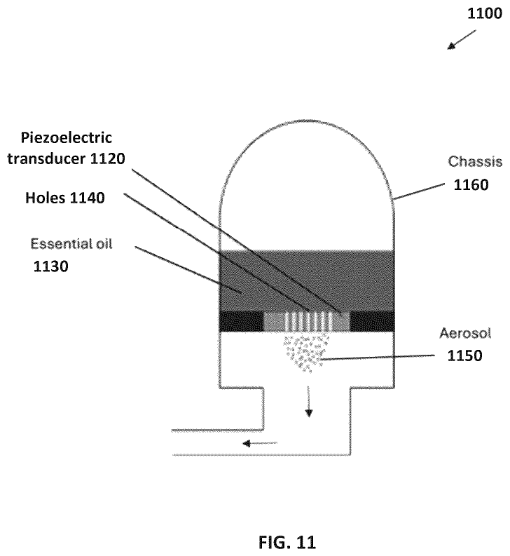

The scent presentation device/system can use a mesh nebulizer as a scent release mechanism. This embodiment, shown in FIG. 11, uses a mesh or membrane with precisely sized openings (holes 1140), through which the liquid 1130 (e.g., essential oil(s) or water-oil mixture) is forced under pressure, creating a fine mist or aerosol. This category of systems can be subdivided into active and passive systems. Active mesh nebulizers employ a vibrating element (e.g., driven by a piezoelectric transducer 1120) to propel the liquid through the mesh, whereas passive systems rely on negative pressure to draw the liquid across. In either case, an aerosol 1150 results. The precision engineering of the mesh ensures consistent droplet size, making these devices highly efficient for delivery with minimal residue.

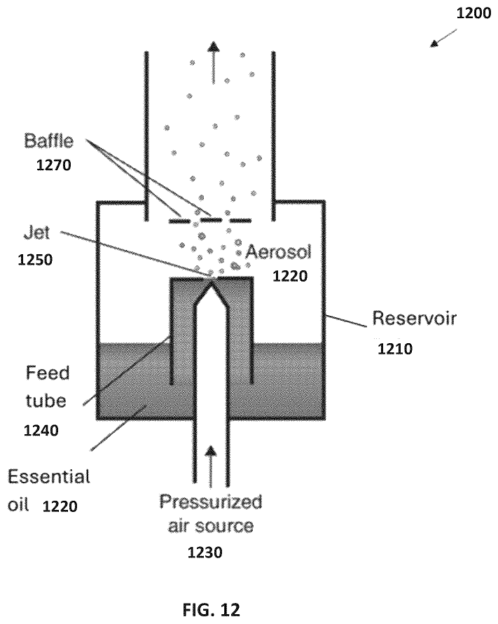

The scent presentation device/system can alternatively use a jet nebulizer as the scent release mechanism as shown in FIG. 12. This operates on the principle of pressurized air flowing through a narrow orifice, converting a liquid into aerosol droplets. This process may involve a Venturi system, where the high velocity of air creates a negative pressure that draws the liquid from the reservoir, atomizing it into a breathable mist. In the figure, a reservoir 1210 holds an essential oil or oil/water mixture 1220. A pressurized air source 1230 pushes air into the device through a feed tube 1240 to produce a jet 1250. This creates a low-pressure area drawing the oil up and creating an aerosol 1260. The aerosol may pass through a baffle 1270 before being released.

A scent presentation device/system can be incorporated into an HVAC system. The dispersion system can be integrated with a building's heating, ventilation, and air conditioning (HVAC) system to disperse scents throughout large areas or entire buildings efficiently. The dispersion system can use cold-air diffusion technology to atomize essential oils into a fine, dry mist that is then distributed evenly through the HVAC ducts. This method is ideal for large spaces such as whole-house, offices, and hotels. Similar to HVAC scenting systems but on a smaller scale, cold-air diffusers also use cold-air diffusion technology to atomize essential oils without heat, preserving their therapeutic properties. These standalone units are suitable for medium to large spaces that require consistent scent coverage without connection to the HVAC system.

In an embodiment, a scent presentation system may be integrated into an HVAC system and controlled by smart home technology. This would allow scent presentation to occur according to a schedule that is defined by a user and programmed into the smart home system. Further, a scent dispersion system may be configured to present a scent, pause presentation, then present a different scent (discussed below). In this case, a fan in the HVAC system may be activated during the interval in which presentation is paused, thereby clearing the first scent before the second scent is presented.

It should be noted that the scent presentation system can use any type of aerosolization and evaporation technologies described herein.

### Pyramid Model

The variety of scents may be modeled as a layered pyramid. The pyramid model may be divided into three main categories or layers of notes, each representing a specific phase of a fragrance's life. These layers are:

Top Notes (Head Notes): These are the initial scents that are perceived immediately upon application of a perfume, for example. Top notes are usually light, fresh, and volatile, evaporating quickly. They give the first impression of the fragrance but last for a short duration, typically ranging from a few minutes to an hour. Common top notes include citrus (like lemon and bergamot), light fruits (such as berries and grapes), and herbs (like lavender and rosemary).

Middle Notes (Heart Notes): After the top notes dissipate, the middle notes become noticeable. They are considered the heart of the fragrance, representing the main elements of the scent composition. Middle notes are more mellow and rounded than top notes and are perceived during the majority of the fragrance's wear on the skin. They typically last longer than top notes but are less volatile, usually lasting several hours. Common middle notes include floral (like rose and jasmine), spice (such as cinnamon and cardamom), and fruit scents (such as peach and plum).

Base Notes: These are the last to develop and the longest-lasting notes. Base notes give depth and solidity to the fragrance, lingering for an extended time after the top and middle notes have faded. They are usually rich, heavy, and deep scents that anchor the fragrance, preventing the lighter notes from evaporating too quickly. Common base notes include woodsy scents (like sandalwood and cedarwood), resins (such as amber and myrrh), musk, and vanilla.

In the above topology of notes, base notes may be described as “lower” than middle notes, which are lower than top notes. Conversely, top notes may be described as “higher” than middle notes, which are higher than base notes.

In some embodiments, a break period may be presented by the scent presentation system between the presentation of different scents. During this break period, no scent is dispersed. The break period can be longer following base notes than middle or top notes, and the break period after a middle note can be longer than that following a top note. The break period can also be based on the room size, number of windows, available air ducts, type of scents, or a combination thereof.

### Control Module

The scent presentation device/system may include a control module to disperse a first scent for a predetermined first duration, employ a break period after the predetermined first duration, and disperse a second scent for a predetermined second duration after the break period. In an embodiment, multiple different scents may be presented in sequence, but separated by break periods. The break period can be based on the type of scent just dispersed, how strong the scent is, and/or where it is located on a scent pyramid model.

The control module may comprise programmable logic circuit (PLC) that allows for the customization of operational sequences. The PLC may be programmed to initiate a control signal to activate a scent release mechanism for a specified first duration.

Upon receiving a control signal, the scent release mechanism may operate for a predetermined first duration, performing its designated function. The duration may be programmable and can be set according to the requirements of a specific application.

After operating for a first duration, the PLC may initiate a break period. This period may be of a predefined or programmable length during which scent release mechanisms are inactive. This break period facilitates the dissipation of the first scent, ensuring it can no longer be detected (via smell).

After the break period, the PLC may generate a second control signal to activate the same or different scent release mechanism to disperse a second scent. The same or different scent release mechanism then operates for a predetermined second duration.

Incorporated within the PLC may be timing and sequencing logic algorithms that ensure the precise control of the activation periods and the break period. This logic is programmable to accommodate various sequences, durations, and operational requirements.

The control module may include an interface for programming and adjusting the sequence parameters, durations, and break periods. This interface may be a physical set of controls on the module or a software application communicating with the module via wired or wireless connections (e.g., over-the-air updates).

### Physical Design

The scent presentation device/system can have a plurality of reservoirs and scent release mechanisms, each supplying a different scent. The plurality of reservoirs can also include one or more scent neutralizers that may be dispersed in the same manner as the essential oils or other scent-bearing liquids described above.

In an embodiment, liquid (e.g., oil, water-oil mixture) having a first scent is transferred into a dispersion reservoir where the scent release mechanism is configured to disperse any liquid transferred into the dispersion reservoir. In this embodiment, the dispersion device/system can have multiple scent reservoirs and a single dispersion reservoir. In another embodiment, the scent presentation device/system can have multiple scent release mechanisms. In this embodiment, each scent reservoir can have its own scent release mechanism and as such a centralized dispersion reservoir is not needed. However, the dispersion system can include a combination of scent reservoirs having their own scent release mechanisms and scent reservoirs with a shared dispersion chamber.

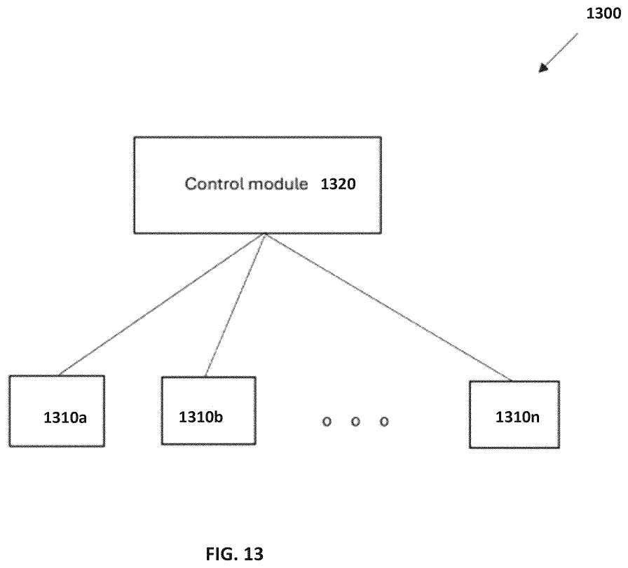

In an embodiment, shown in FIG. 13, the plurality of scent release mechanisms 1310 may be under the control of a control module 1320. Each scent release mechanism may disperse different scents. The control module may control which scent release mechanism is operating at a given time, when it starts, when it stops, and how long a break period lasts before another scent release mechanism, with a different scent, begins.

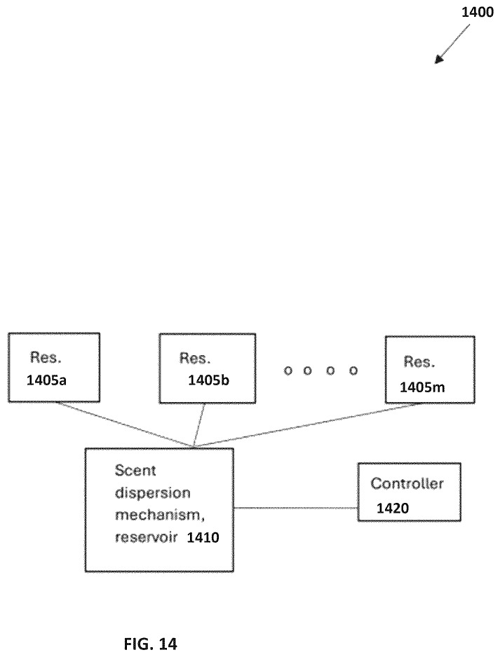

In some embodiments, the device/system can have multiple reservoirs, where the system also includes a scent release mechanism having a dispersion reservoir configured to disperse any liquid in the dispersion reservoir. This is shown in FIG. 14. The scent release mechanism with its reservoir (910) can be centrally located to the multiple scent reservoirs 1405. In operation, liquid from one of the scent reservoirs can be transferred (e.g., pump, gravity) into the dispersion reservoir. Once the transfer process is completed, the scent release mechanism can be activated to disperse the liquid therein. As before, a control module (here, 1420) may be used to control which scent reservoir (i.e., which scent) is being used at a given time, when the dispersion of that scent starts, when it stops, and how long a break period lasts before a different scent begins.

### Further Embodiments

In some embodiments, liquid from two or more reservoirs can be transferred to the dispersion reservoir for dispersion. In this way, a unique blend of aromas can be created using two or more scented liquids.

In some embodiments, the dispersion device/system can dispense droplets of the liquid (e.g., essential oil, water-oil mixture) into a dispersion chamber where the droplets are exposed to an airstream using various means such as a fan, convection, pressure differential system, etc. The dispersion chamber may also include a dispersion plate configured to disperse or evaporate the droplets. The dispersion plate can employ aerosolization or evaporation mechanism as described herein. For example, the dispersion plate can use an ultrasonic transducer to disperse the droplets. Alternatively, the dispersion plate can use a heat source to speed up the evaporation process.

Various mechanisms can be employed to accurately disperse liquid droplets. The dispersion device/system can employ any of the droplets dispensing mechanisms described herein.

Piezoelectric droplet ejection leverages the properties of piezoelectric materials to precisely eject liquid droplets, a technique widely adopted in inkjet printing. Similarly, microfluidic devices manipulate fluids within micro-scale channels to produce uniform droplets.

As noted above, the sequence of scents as presented to the user may or may not coincide with the arrangement of scent reservoirs on the belt. After a determined scent has been presented to the user, the next scent on the schedule may not correspond to the adjacent scent reservoir on the belt. In either case, the belt will be rotated until the reservoir having the next scent is positioned at the opening of the lower drum. The rotation of the belt to the correct position may be facilitated by the operation of the belt position sensor and the position indicators on the belt.

It can be desirable to isolate a given scent for presentation to a user, in that unintentional mixing of scents may diminish the therapeutic value of the apparatus and its operation. Preferably, a scent may be presented to a user, perhaps followed by an interval in which the scent dissipates prior to a next scent being presented. In an embodiment, this isolation may be facilitated by introducing an intervening odor that serves as a separator between successive scents. Such an intervening odor may serve to neutralize a preceding scent. Compounds that may be used for this purpose include hydroxypropyl cyclodextrin, diethylene glycol, sodium citrate, sodium maleate, or a combination of some or all of these. Alternatively, the intervening odor may be a mixture of odors that in combination create an olfactory white odor.

To implement such an intervening odor, one or more scent reservoirs 150 may hold scent source material that comprises such a mixture of odors, or one or more of the above compounds. Such scent source material may or may not be embodied in wax. The intervening odor may then be included in the schedule programmed into the logic of the control module. The apparatus would then present a scent from a particular scent reservoir, followed by presentation of an intervening odor from its scent reservoir, followed by presentation of another scent, etc. In various embodiments, presentation of an intervening odor may or may not require heating.

As noted above, in an embodiment the apparatus may include one or more doors that seal off the chamber while a reservoir is heated. After a predetermined period during which the scent has accumulated in the closed chamber, the doors are opened and the fan expels the accumulated scent. While the fan is activated and the doors are open, the heating may stop; in other embodiments, the heating may continue while the doors are open. This sequence—heating with the doors closed followed by opening the doors and expelling the scent—may be performed one or more times while a reservoir is in place adjacent to the heating element. After heating of the reservoir is completed and the dispersion of its scent is concluded, the belt may be shifted so that the next scheduled reservoir is placed adjacent to the heating element. The sequence of heating this next reservoir with the doors closed, then opening the doors and expelling the scent, may then be performed one or more times with this reservoir. In this manner, scents from the various reservoirs may be presented to a user in relatively concentrated bursts.

As in other embodiments, the schedule of scents may be determined by the programmed control module. The duration of the heating with the doors closed may also be controlled by the control module; the duration of scent expulsion with the doors open may also be controlled by the control module. The number of times that the heating and expulsion are performed while a given reservoir is in place may also be controlled by the control module.

Any or all of these parameters—the duration of initial scent release, the duration of scent expulsion, and the number of times that the heating and expulsion are performed for a given reservoir—may be different for different reservoirs and scents. For example, a milder scent such as vanilla may have a longer heating duration than other scents, so that more of this scent may be accumulated in the closed chamber before the scent is released.

In an embodiment, the burst expulsion of a scent may be followed by the normal operation previously described. For example, once a reservoir is in place, the doors to the chamber may be closed as the reservoir is heated and its scent is released into the chamber. After a predetermined interval, the doors may be opened and the accumulated scent may be pushed out of the chamber by the fan. In this embodiment, the heating may continue while the doors remain open and the fan continues to operate, so that the scent continues to be dispersed through the chamber.

Eventually the heating stops, the reservoir is allowed to cool, and the belt is turned so that the reservoir having the next scheduled scent is now adjacent the heating element. The process may then repeat for this scent: The doors may be closed, and this next reservoir may be heated while the user experiences his break period during which no scent is presented. This next scent accumulates in the closed chamber as its reservoir is heated. After a predetermined period, the chamber doors are opened and the fan pushes the accumulated scent out of the chamber. As before the heating may continue while the doors remain open and the fan continues to operate, so that the scent continues to be released from the chamber. The process may repeat for any or all scents in the schedule.

While the above embodiments perform expulsion of an accumulated scent in a burst fashion, this process and mechanism may be implemented in an apparatus other than the belt-based design discussed above. Other mechanisms may alternatively be used to hold and position a plurality of scent reservoirs. In such alternative mechanisms, the burst expulsion process may still be implemented using an airflow chamber that may be opened and closed as described above.

As discussed above, alternative embodiments may include different mechanisms for initial scent release from a source, different from the heating of a scent reservoir. For example, other embodiments can use mechanisms such as a diffusion mechanism, scent release mechanism, nebulization mechanism, evaporative mechanism, or a combination thereof, for example and without limitation.

With any of these alternative mechanisms, the scent may be initially released into a closed airflow chamber where it may be allowed to accumulate. After a predetermined period, the airflow chamber may be opened and the accumulated scent presented in a burst to a user through use of a fan or other airflow mechanism.

In such alternative embodiments, the burst dispersion process may still be implemented using an airflow chamber that may be opened and closed as described above.

Some embodiments of the present invention have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

What is claimed is:

1. A scent presentation system, comprising:
a plurality of scent reservoirs, each scent reservoir containing a scent source material associated with a respective odor;
an isolation chamber configured to receive vapor from a selected one of the scent reservoirs while isolating remaining ones of the scent reservoirs from the isolation chamber;
a positioning mechanism configured to selectively place individual scent reservoirs in fluid communication with the isolation chamber;
a scent release mechanism configured to cause release of vapor from the selected scent reservoir into the isolation chamber;
an airflow mechanism configured to expel vapor from the isolation chamber toward a user;
one or more doors associated with the isolation chamber and movable between a closed configuration in which vapor accumulates within the isolation chamber and an open configuration in which accumulated vapor is expelled as a burst; and
a control module configured to control the positioning mechanism, the scent release mechanism, the one or more doors, and the airflow mechanism according to a programmed scent presentation schedule.

2. The system of claim 1, wherein the positioning mechanism comprises a belt carrying the plurality of scent reservoirs.

3. The system of claim 2, wherein the belt is configured as a closed loop movable around at least one drum.

4. The system of claim 1, wherein the isolation chamber is defined at least in part by a stationary drum having an opening aligned with the selected scent reservoir.

5. The system of claim 1, wherein each scent reservoir contains an odor-infused wax material.

6. The system of claim 5, wherein the scent release mechanism comprises a heating element configured to raise a temperature of the odor-infused wax material above a melting temperature.

7. The system of claim 6, wherein the heating element is movable between a heating position adjacent the selected scent reservoir and a retracted position spaced from the selected scent reservoir.

8. The system of claim 1, wherein each scent reservoir includes a membrane configured to permit passage of vapor while retaining solid or molten scent material.

9. The system of claim 1, further comprising a position sensor configured to provide information identifying which scent reservoir is in fluid communication with the isolation chamber.

10. The system of claim 9, wherein the position sensor detects at least one identifying marker associated with a scent reservoir or with the positioning mechanism.

11. The system of claim 1, wherein the control module is programmable to specify at least one of a vapor accumulation duration, a vapor expulsion duration, a number of burst events, a heating duration, or a cooling duration for individual scent reservoirs.

12. The system of claim 1, wherein the scent presentation schedule includes presentation of an intervening odor configured to reduce perceptibility of a preceding odor.

13. The system of claim 12, wherein the intervening odor is produced from a scent reservoir containing a scent neutralizing compound.

14. A method of presenting a scent to a user, comprising:
determining, by a control module, a scent to be presented according to a programmed schedule;
positioning a scent reservoir associated with the determined scent in fluid communication with an isolation chamber; and
heating the scent reservoir to release the scent into the isolation chamber while operating an airflow mechanism to expel the released scent toward the user.

15. The method of claim 14, wherein the heating is maintained until a heating interval determined by the control module is completed and further comprising terminating the heating to allow cooling of the scent reservoir, wherein cooling of the scent reservoir is maintained until a cooling interval determined by the control module is completed.

16. The method of claim 14, further comprising closing one or more doors associated with the isolation chamber to accumulate vapor during at least a portion of the heating, and opening the one or more doors while operating the airflow mechanism to expel accumulated vapor as a burst.

17. A scent presentation system, comprising:
a belt carrying a plurality of scent reservoirs, each scent reservoir associated with a respective odor;
an isolation chamber selectively alienable with an individual scent reservoir on the belt; a positioning mechanism configured to index the belt such that a selected scent reservoir is placed in fluid communication with the isolation chamber while remaining scent reservoirs are isolated;
a scent release mechanism configured to cause release of scent from the selected scent reservoir into the isolation chamber;
an airflow mechanism configured to expel scent from the isolation chamber toward a user;
one or more doors associated with the isolation chamber and movable between a closed configuration in which scent accumulates within the isolation chamber and an open configuration in which accumulated scent is expelled as a burst; and
a control module configured to control belt positioning, operation of the scent release mechanism, movement of the one or more doors, and operation of the airflow mechanism according to a programmed scent presentation schedule.

18. The system of claim 17, wherein the belt is configured as a closed loop movable around at least one drum.

19. The system of claim 17, wherein the control module is configured to perform multiple burst expulsion events for a single scent reservoir while the scent reservoir remains aligned with the isolation chamber.

20. The system of claim 17, wherein the control module is configured to vary at least one of an accumulation duration, a burst duration, or a fan speed based on characteristics of the selected scent.
